(19) United States
(12) Patent Application Publication
(10) Pub. No.: US 2024/0169638 A1
(43) Pub. Date: May 23, 2024

(54) IMAGE PROCESSING APPARATUS, IMAGE PROCESSING METHOD, AND RECORDING MEDIUM

(71) Applicant: SONY GROUP CORPORATION, TOKYO (JP)

(72) Inventor: YOSHINORI OHASHI, TOKYO (JP)

(21) Appl. No.: 18/551,204

(22) PCT Filed: Jan. 25, 2022
(86) PCT No.: PCT/JP2022/002510

§ 371 Date: Sep. 19, 2023

(30) Foreign Application Priority Data
Mar. 31, 2021 (JP) 2021-059301

(51) Int. Cl. G06T 13/40; A63B 71/06; G06T 7/73; G06T 15/20; G06T 19/00

(52) U.S. Cl. CPC G06T 13/40; A63B 71/0619; G06T 7/73; G06T 15/20; G06T 19/00; G06T 2207/30196; G06T 2219/012

(57) ABSTRACT

The present disclosure relates to an image processing apparatus and an image processing method as well as a recording medium by which distribution of content that can be displayed from a free viewpoint can be performed favorably in real time. A deciding unit estimates, in a case where, according to a result of comparison in corresponding relation between multiple pieces of joint information of an imaging target estimated from a captured image and multiple pieces of character model joint information of a desired character model, the multiple pieces of joint information and the multiple pieces of character model joint information are incoincident at least in part thereof, the character model joint information having no corresponding relation with the multiple pieces of joint information and decides all of the pieces of character model joint information. A posture estimation unit estimates a posture of the whole body of the character model, by using as an initial position, the decided character model joint information. The present technology can be applied, for example, to a distribution system that performs real time free viewpoint distribution.

### TECHNICAL FIELD

The present disclosure relates to an image processing apparatus and an image processing method as well as a recording medium, and particularly to an image processing apparatus and an image processing method as well as a recording medium by which distribution of content that can be displayed in real time from a free viewpoint can be performed favorably.

### BACKGROUND ART

Conventionally, development of a technology for distributing content that estimates joint positions of the body of a sports player from an image and applies the estimated joint positions to a desired 3D character model to make it possible to display a sports competition in real time from a free viewpoint has been progressed.

In order to apply a joint position estimated from an image to a desired 3D character model in such a manner, it is necessary to appropriately determine not only joint positions but also rotation of all bones. However, since rotation of bones differs depending upon a 3D character model, it is difficult to apply joint positions estimated from an image to a desired 3D character model and draw the 3D character model in a natural posture.

Hence, for example, as disclosed in PTL 1, there has been developed a technology which applies expanded FABRIK (Forward And Backward Reaching Inverse Kinematics) to a tree structure in which joint positions are inputted, to appropriately determine rotation of individual bones.

### CITATION LIST

### Patent Literature

### [PTL 1]

PCT Patent Publication No. WO2020/070928

### SUMMARY

### Technical Problem

Incidentally, in the technology proposed in PTL 1 described above, in a case where a joint position existing in a 3D character model does not exist at an inputted joint position, some difficulty may occur with drawing of the 3D character model in a natural posture in some cases. Hence, in such a case as just described, it is supposed that, even if content to be distributed is displayed in real time from a free viewpoint, the display becomes unnatural and distribution of such content as just described cannot be performed favorably.

The present disclosure has been made in view of such a situation as described above and makes it possible to favorably perform distribution of content that can be displayed in real time from a free viewpoint.

### Solution to Problem

An image processing apparatus of one aspect of the present disclosure includes a deciding unit that estimates, in a case where, according to a result of comparison in corresponding relation between multiple pieces of joint information of an imaging target estimated from a captured image and multiple pieces of character model joint information of a desired character model, the multiple pieces of joint information and the multiple pieces of character model joint information are incoincident at least in part thereof, any one or more of the multiple pieces of character model joint information having no corresponding relation with the multiple pieces of joint information and decides all of the relevant pieces of character model joint information, and a posture estimation unit that estimates a posture of the whole body of the character model, by using as initial positions, the decided relevant pieces of character model joint information.

An image processing method or a recording medium of the one aspect of the present disclosure includes estimating, in a case where, according to a result of comparison in corresponding relation between multiple pieces of joint information of an imaging target estimated from a captured image and multiple pieces of character model joint information of a desired character model, the multiple pieces of joint information and the multiple pieces of character model joint information are incoincident at least in part thereof, any one or more of the multiple pieces of character model joint information having no corresponding relation with the multiple pieces of joint information and deciding all of the relevant pieces of character model joint information, and estimating a posture of the whole body of the character model, by using as initial positions, the decided relevant pieces of character model joint information.

In the one aspect of the present disclosure, in a case where, according to a result of comparison in corresponding relation between multiple pieces of joint information of an imaging target estimated from a captured image and multiple pieces of character model joint information of a desired character model, the multiple pieces of joint information and the multiple pieces of character model joint information are incoincident at least in part thereof, any one or more of the multiple pieces of character model joint information having no corresponding relation with the multiple pieces of joint information are estimated and all of the relevant pieces of character model joint information are decided. Then, a posture of the whole body of the character model is estimated, by using as initial positions, the decided relevant pieces of character model joint information.

### BRIEF DESCRIPTION OF DRAWINGS

FIG. 1 is a block diagram depicting an example of a configuration of an embodiment of a distribution system to which the present technology is applied.

FIG. 2 is a block diagram depicting an example of a configuration of a client terminal.

FIG. 3 is a diagram depicting an example of a skeleton structure to be applied to a 3D character model.

FIG. 4 is a view illustrating an example of a process in the client terminal.

FIG. 5 is a view illustrating an example of application to 3D character models that are each three heads tall and have different number of joints.

FIG. 6 is a flow chart illustrating a real time free viewpoint reproduction process.

FIG. 7 is a flow chart illustrating a forward reach phase process.

FIG. 8 is a flow chart illustrating a rearward reach phase and root group deformation process.

FIG. 9 is a block diagram depicting an example of a configuration of an embodiment of a computer to which the present technology is applied.

### DESCRIPTION OF EMBODIMENT

In the following, a specific embodiment to which the present technology is applied is described in detail with reference to the drawings.

### Example of Configuration of Distribution System

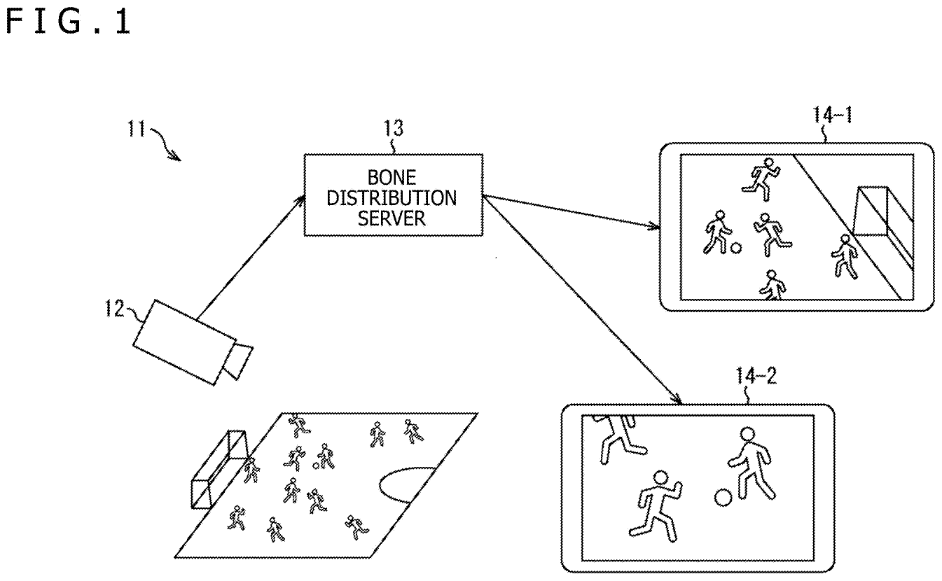

FIG. 1 is a block diagram depicting an example of a configuration of an embodiment of a distribution system to which the present technology is applied.

A distribution system 11 depicted in FIG. 1 is configured such that a capture system 12, a bone distribution server 13, and multiple client terminals 14 (in the example of FIG. 1, two client terminals 14-1 and 14-2) are connected to each other through a network. Here, described is an example of use in which the distribution system 11 is used for distribution (hereinafter referred to as real time free viewpoint distribution) of content by which a sports competition can be displayed in real time from a free viewpoint. It is to be noted that the distribution system 11 can be used for real time free viewpoint distribution of not only sports but also various kinds of content.

The capture system 12 includes multiple capturing apparatuses and captures a sports competition from various directions by the capturing apparatuses to acquire multiple moving images. Further, the capture system 12 analyzes the multiple moving images and transmits joint position information acquired by estimating joint positions of a sports player to the bone distribution server 13 through a network.

For example, the joint position information includes a list of data, each data including a person ID (Identification) for identifying a person such as a sports player, a joint ID for identifying a joint that can be estimated by the capture system 12, and position data (x, y, z) indicative of a position of a joint for each joint ID. Hence, the joint position information is transmitted with a data amount smaller than that in transmission of, for example, a moving image.

The bone distribution server 13 receives the joint position information transmitted thereto from the capture system 12 and distributes the joint position information in real time to the multiple client terminals 14 through the network.

The client terminal 14 applies to a desired 3D character model the joint position information distributed thereto from the bone distribution server 13, to duplicate an actual posture of a sports player on the 3D character model. Then, the client terminal 14 performs rendering of the 3D character model duplicating the posture of the sports player, for example, in such a manner that a sports competition is being enjoyed from a free viewpoint according to an operation of a user. Accordingly, as depicted in FIG. 1, a user of the client terminal 14-1 and a user of the client terminal 14-2 can enjoy watching a sports competition from viewpoints different from each other. The client terminal 14 may download and use, for example, a 3D character model stored in a cloud or may use a 3D character model in a local environment of the user. Further, the client terminal 14 may use a two-dimensional character model in place of a 3D character model.

In such a manner, the distribution system 11 can implement real time free viewpoint distribution by such a configuration that joint position information of a sports player is distributed and the client terminal 14 applies the joint position information to a desired 3D character model, and the user can enjoy watching a sports competition with the 3D character model that duplicates the posture of the sports player being changed over freely.

Incidentally, even if a sports competition is captured from multiple directions in the capture system 12, for example, in a case where an arm, a foot, or the like is hidden by the body of a sports player, it sometimes occurs that all joint positions cannot be estimated. In particular, multiple joints estimated from a sports player captured in an image and multiple joints configuring the skeleton structure of the 3D character model become incoincident with each other. It is supposed that, in this case, joint position information in which the joint position that has not been estimated successfully and is missing is distributed to the client terminal 14, and, even if joint position information in which the greatest number of joint positions are estimated is applied to the 3D character model and rendering is performed, it is difficult to duplicate the actual posture of the sports player on the 3D character model.

Hence, the distribution system 11 is configured such that, even in a case where joint position information in which a joint position is missing is distributed, the client terminal 14 can duplicate the actual posture of the sports player on a 3D character model. It is to be noted that, in the distribution system 11, joint information indicative of the number of joints and joint positions of an imaging target estimated from a captured image may be distributed.

### Example of Configuration of Client Terminal

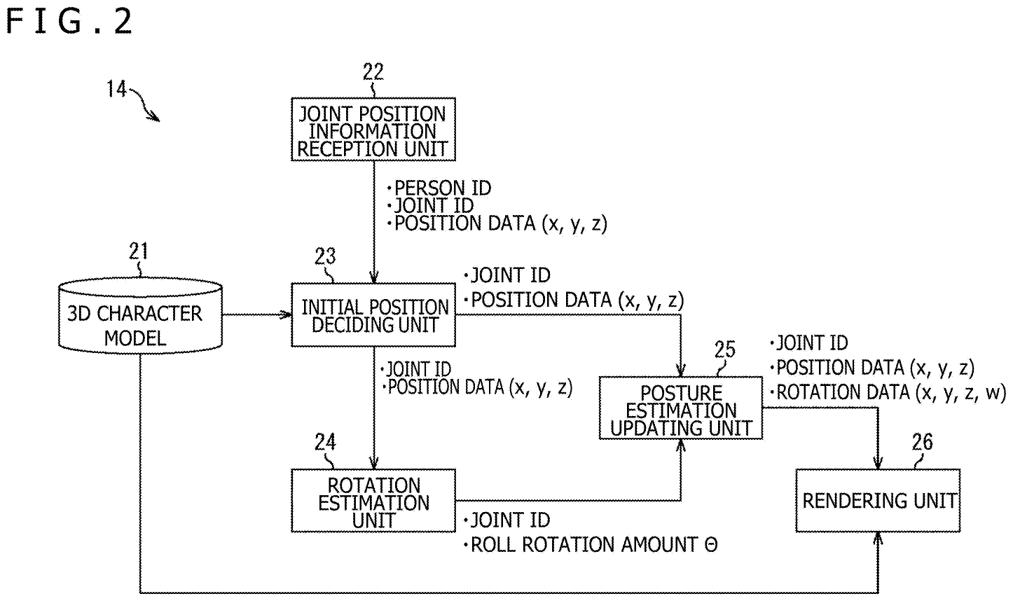

FIG. 2 is a block diagram depicting an example of a configuration of the client terminal 14.

As depicted in FIG. 2, the client terminal 14 includes a 3D character model storage unit 21, a joint position information reception unit 22, an initial position deciding unit 23, a rotation estimation unit 24, a posture estimation updating unit 25, and a rendering unit 26.

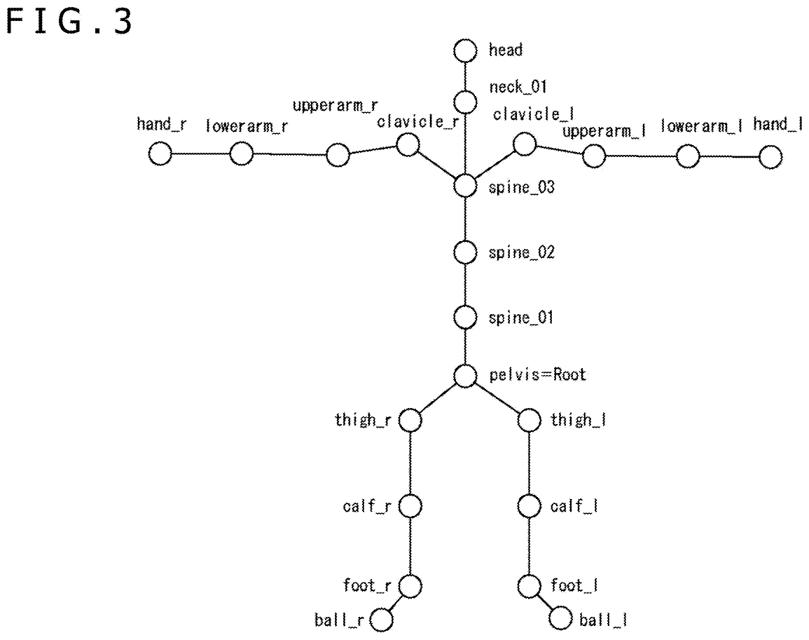

The 3D character model storage unit 21 has desired 3D character models stored therein. For example, the 3D character models have a three-dimensional skeleton structure defined for individual 3D character models. In FIG. 3, an example of the skeleton structure applied to a 3D character model stored in the 3D character model storage unit 21 is depicted. Further, the skeleton structure of such a shape as depicted in FIG. 3 is used as a basic structure of the 3D character model. By joint position information being applied to such a skeleton structure as just described, the body shape of the 3D character model is adjusted to coincide with the body shape of a sports player.

Here, the skeleton structure is configured such that multiple joint nodes are connected to each other by bones. In the example depicted in FIG. 3, the skeleton structure has a tree structure in which a joint node pelvis of the pelvis is a root node. It is to be noted that, in addition to the joint node pelvis of the pelvis, joint nodes spine_1 to spine_3 of the spine may be treated as root nodes. Further, each of the joint nodes other than the root nodes (the joint node head of the head, the joint node neck_1 of the neck, the joint node clavicle_r of the right clavicle, the joint node upperarm_r of the right upper arm, the joint node lowerarm_r of the right forearm, the joint node hand_r of the right hand, the joint node clavicle_1 of the left clavicle, the joint node upperarm_1 of the left upper arm, the joint node lowerarm_1 of the left forearm, the joint node hand_1 of the left hand, the joint node thigh_r of the right thigh, the joint node calf_r of the right calf, the joint node foot_r of the right foot, the joint node ball_r of the base of the right toe, the joint node thigh_r of the left thigh, the joint node calf_r of the left calf, the joint node foot_r of the left foot, and the joint node ball_r of the base of the left toe) is also referred to as an end joint node.

The joint position information reception unit 22 receives joint position information distributed thereto from the bone distribution server 13 and supplies the joint position information to the initial position deciding unit 23. For example, the joint position information reception unit 22 supplies a list of joint IDs and position data (x, y, z) associated individually with person IDs of all sports players participating in a sports competition to the initial position deciding unit 23. In the following description, it is assumed that processes executed by the client terminal 14 are performed for each person ID.

The initial position deciding unit 23 decides initial positions of all joints configuring the skeleton structure of a 3D character model, by using the joint IDs and the position data (x, y, z) supplied from the joint position information reception unit 22. At this time, in a case where any joint is missing in the joint position information, the initial position deciding unit 23 can estimate the position of the joint of the 3D character model which is missing in the joint position information.

For example, the initial position deciding unit 23 compares multiple joints identified by the joint IDs included in the joint position information supplied from the joint position information reception unit 22 and multiple joints configuring the skeleton structure of the 3D character model stored in the 3D character model storage unit 21 with each other to specify a corresponding relation between the joints. Then, in regard to any joint that is among the multiple joints configuring the skeleton structure of the 3D character model and whose corresponding relation with the multiple joints identified by the joint IDs of the joint position information has been specified successfully, the initial position deciding unit 23 decides to use the position data (x, y, z) of the joint position information as an initial position of the specified joint.

On the other hand, in a case where the multiple joints configuring the skeleton structure of the 3D character model include a joint whose corresponding relation with the multiple joints identified by the joint IDs of the joint position information cannot be specified, the initial position deciding unit 23 decides that a joint is missing in the joint position information. Accordingly, in this case, the initial position deciding unit 23 estimates the position of the joint of the 3D character model missing in the joint position information and decides to use the position data (x, y, z) of the estimated position as an initial position of the joint missing in the joint position information.

In such a manner, the initial position deciding unit 23 can decide the initial positions of all joints configuring the skeleton structure of the 3D character model, and supplies a list of the joint IDs and the position data (x, y, z) of the joints to the rotation estimation unit 24 and the posture estimation updating unit 25.

The rotation estimation unit 24 estimates, in reference to the joint IDs and the position data (x, y, z) supplied from the initial position deciding unit 23, rotation of end joint nodes (for example, the head, both hands, both feet and so forth) on multiple branches and determines a roll rotation amount θ for each joint ID. Further, the rotation estimation unit 24 estimates rotation of root nodes (for example, the waist, breast, and so forth) from which end joint nodes are branched and determines the roll rotation amount θ for each joint ID. Thus, the rotation estimation unit 24 determines the roll rotation amount θ of all joint nodes of the 3D character model and supplies the joint IDs and the roll rotation amounts θ to the posture estimation updating unit 25.

The posture estimation updating unit 25 receives, as inputs thereto, the joint IDs and the position data (x, y, z) supplied from the initial position deciding unit 23 and the joint IDs and the roll rotation amounts θ supplied from the rotation estimation unit 24 and executes the FABRIK Tree algorithm to estimate the posture of the whole body of the 3D character model. It is to be noted that the FABRIK Tree algorithm is disclosed in detail in PTL 1 described hereinabove, and the substance of PTL 1 is incorporated in the present embodiment such that the posture estimation updating unit 25 can execute the FABRIK Tree algorithm. Further, the posture estimation updating unit 25 supplies the position data (x, y, z) and the rotation data (x, y, z, w) for each joint ID determined as a result of the estimation of the posture of the whole body of the 3D character model to the rendering unit 26.

The rendering unit 26 reads out the 3D character model from the 3D character model storage unit 21 and applies the position data (x, y, z) and the rotation data (x, y, z, w) supplied from the posture estimation updating unit 25, to the joint nodes of the skeleton structure configuring the 3D character model. The rendering unit 26 thereby duplicates the actual posture of the sports player on the 3D character model, renders the 3D character model from a desired free viewpoint to draw an image of the sports competition, and outputs the drawn image to a display device not depicted. Further, the rendering unit 26 preserves the position data (x, y, z) and the rotation data (x, y, z, w) such that, when the sports competition is to be watched again, the rendering unit 26 can apply the posture to a 3D character model having the same skeleton and re-render the 3D character model without carrying out the FABRIK Tree algorithm.

The client terminal 14 configured in such a manner can draw a 3D character model in a natural posture by estimating, even if any joint is missing in the joint position information distributed from the bone distribution server 13, the position of the missing joint to determine its initial position.

Accordingly, in the distribution system 11, even if content is displayed in real time from a free viewpoint, it can be avoided that the display becomes unnatural, and distribution of content that can be displayed in real time from a free viewpoint can be performed favorably. Further, the distribution system 11 can be applied also to other than watching of sports. For example, the distribution system 11 may distribute joint information of all performers on a stage in various live performances of a concert, a play, a dance, ballet, kabuki, circus, rakugo, a comedy skit, or the like. Consequently, on the client terminal 14, such various live performances are displayed in real time from a free viewpoint.

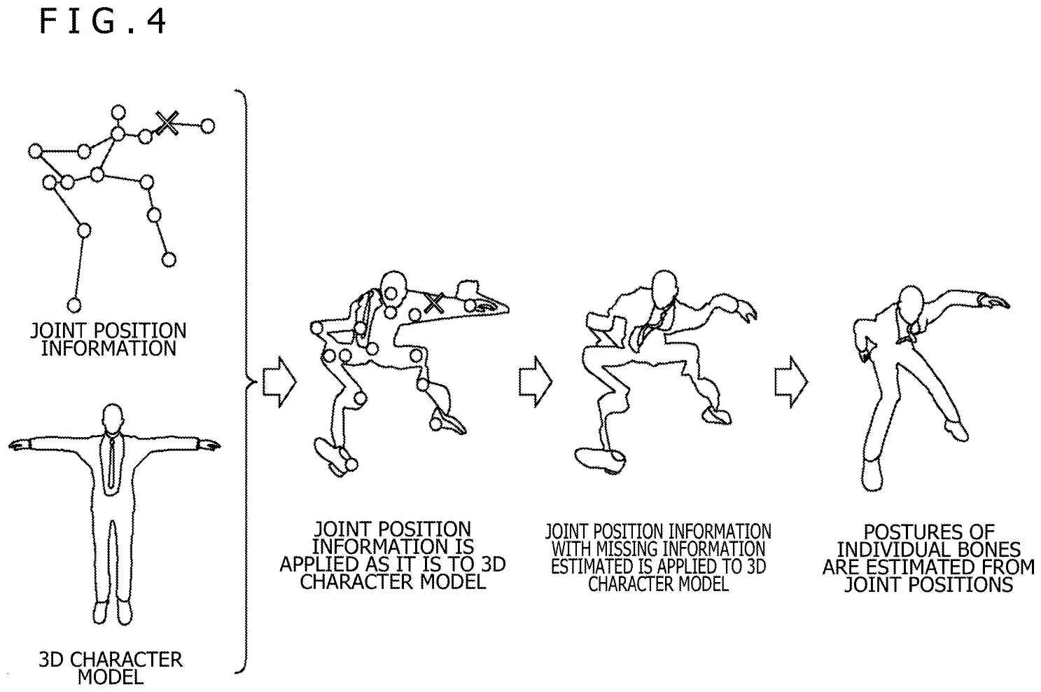

Here, an example of a process by the client terminal 14 of applying, to a 3D character model, joint position information distributed from the bone distribution server 13 and estimating the posture of individual bones of the skeleton structure configuring the 3D character model is described with reference to FIG. 4.

For example, while joint position information distributed from the bone distribution server 13 includes position data (x, y, z) of individual joints, it does not include a rotation amount of the joints. Further, as described hereinabove, in a case where the capture system 12 fails to estimate any joint position, joint position information in which position data of the joint position is missing is distributed. Alternatively, even if the capture system 12 succeeds in estimating the joint position, also in a case where the number of joints of the skeleton structure configuring the 3D character model is great, the joint position information distributed may have any position data of the joint positions missing. For example, in regard to a joint position marked by a cross in the joint position information depicted in FIG. 4, it is represented that position data is missing.

Further, if such joint position information in which position data is missing as described above is applied as it is to a 3D character model, the 3D character model becomes unnatural in regard to the joint of the missing position data. Hence, the client terminal 14 can appropriately determine rotation of all bones configuring the 3D character model, by receiving joint positions of a person as an input thereto, deciding, in order to apply the joint positions to a desired 3D character model and appropriately draw the 3D character model, the joint position missing in the joint position information, as an initial position, and then using the FABRIK algorithm corresponding to the tree structure. The estimation process described above can also be applied to a case in which the joint positions of the 3D character model are smaller in number than those in the joint position information distributed from the bone distribution server 13. In short, in a case where the number of joints included in the joint position information distributed from the bone distribution server 13 is greater than those of the joint positions of the 3D character model, it is sufficient if the positions of only the joints that exist in the 3D character model (whose corresponding relation is specified) and that are included in the distributed joint position information are given as initial positions.

The initial position deciding unit 23 estimates, as an initial position of a joint position that is missing in the joint position information, a missing joint position by disposing, for example, if the joint position is that of a joint node between the waist and the chest, the joint position in a spaced relation by a length of each bone in the basic posture on a straight line interconnecting the waist to the chest. Alternatively, the initial position deciding unit 23 may estimate, as the missing joint position, a joint position included in the joint position information that is immediately preceding the joint position information in which the joint position is missing and that is among the pieces of the joint position information distributed successively from the bone distribution server 13.

Then, the posture estimation updating unit 25 inputs the joint positions to the FABRIK algorithm compatible with a tree structure and performs a forward reach phase and a rearward reach phase repeatedly to merge the joint positions into the 3D character model. Thus, the posture estimation updating unit 25 estimates the posture of the bones from the joint positions, and, for example, determines rotation of all bones and applies them to the 3D character model.

By this, even if any joint position is missing in the joint position information distributed from the bone distribution server 13, the client terminal 14 can appropriately apply the joint position information to a desired 3D character model and perform drawing. Further, even in a case where the joint position information distributed from the bone distribution server 13 does not coincide with the joints of the 3D character model or in a case where the 3D character model is different in body proportion or physique, it is possible to appropriately apply the joint position information to the 3D character model and perform drawing. For example, in the client terminal 14, in a case where the length between joints according to the joint position information and the length between corresponding joints according to the joint positions of the 3D character model are different from each other, the initial position deciding unit 23 decides an initial position after adjusting the length between the joints in the joint position information to the length between the joints of the 3D character model. It is to be noted that, since FABRIK is a lightweight algorithm, it is possible for the client terminal 14 to perform real time processing.

Further, the client terminal 14 can determine rotation, which cannot be determined by FABRIK, of an end joint node or of a root node of the waist or the chest from a positional relation of other joint nodes and use the rotation as an input of FABRIK.

For example, the rotation estimation unit 24 can determine the orientation of the face from the positions of the left and right eyes and can determine the orientation of the feet according to the orientation of the waist. Further, the rotation estimation unit 24 can determine the orientation of the waist from the position of the chest, the right foot, and the left foot and can determine the orientation of the chest from the position of the head, the right shoulder, and the left shoulder.

Further, while the posture of both hands can be determined by calculation in a case where the joints (or equivalents) of two or more fingers can be acquired, in a case where joints of two or more fingers cannot be acquired, the posture of both hands can be determined from the positional relation between the hands and the wrists except roll rotation of both hands (rotation of the arms around the bone axes). For example, roll rotation of both hands can be determined by setting the palms of the right hand and the left hand to normally face each other. Further, in a case where information concerning a ball can be acquired from data for sports, roll rotation of both hands may be determined such that the palm nearest to the position of the ball is directed toward the ball. It is to be noted that a machine learning network that receives position information of other joint nodes of the body as an input thereto or like means may be used.

Further, the client terminal 14 may determine a scale of a 3D character model to be applied, from a maximum value of the distance between joint positions from the head to the feet, and apply the scale. This makes it possible to apply the scale to 3D character models that are three heads tall but are different in joint number.

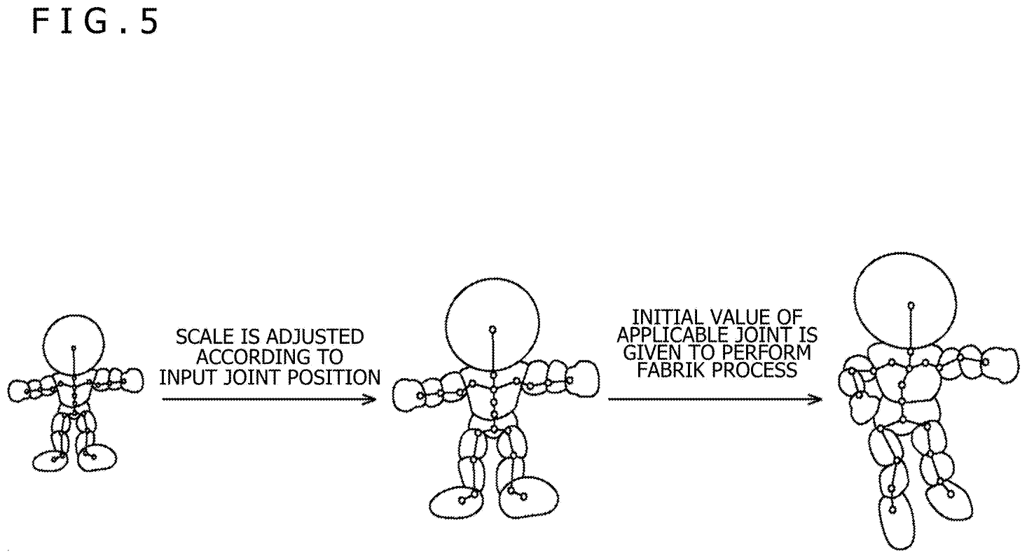

An example of application to 3D character models that are three heads tall but are different in joint number is described with reference to FIG. 5.

For example, in the client terminal 14, the scale of a 3D character model is adjusted such that the distance from the head to the feet of the 3D character model coincides with a maximum value of the distance from the head to the feet among the input joint positions. Then, the initial position deciding unit 23 decides joint position information distributed from the bone distribution server 13, as an initial position, and the posture estimation updating unit 25 executes the FABRIC Tree algorithm by using the initial position as an input thereto. This makes it possible for the client terminal 14 to estimate the posture of the whole body on which the input joint positions are reflected, even where the 3D character model is different in joint number or body proportion.

### Example of Processing of Real Time Free Viewpoint Reproduction Process

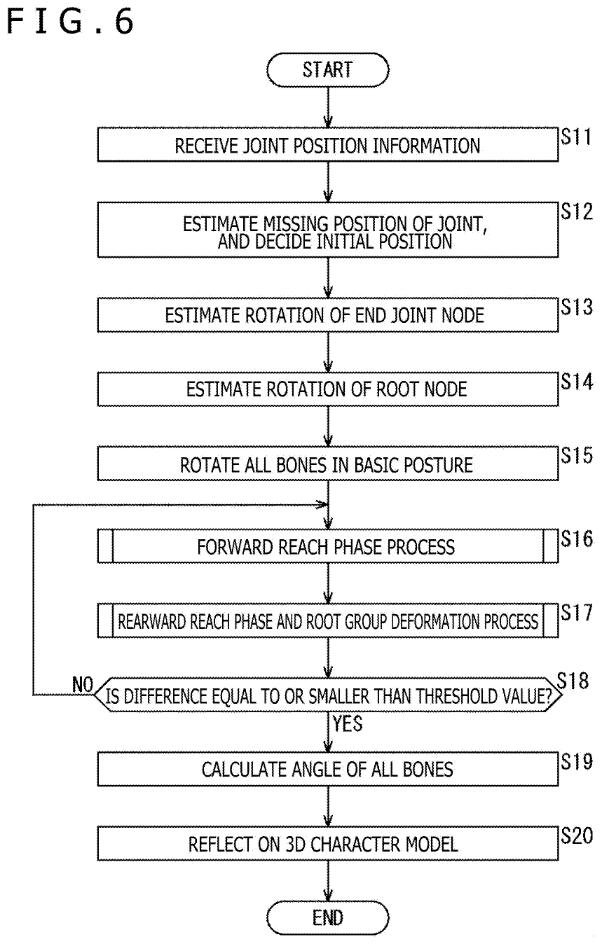

FIG. 6 is a flow chart illustrating a real time free viewpoint reproduction process by the client terminal 14 reproducing content distributed by real time free viewpoint distribution by the distribution system 11.

In step S11, the joint position information reception unit 22 receives joint position information distributed thereto from the bone distribution server 13 and supplies the joint position information to the initial position deciding unit 23.

In step S12, the initial position deciding unit 23 compares multiple joints identified by the joint IDs of the joint position information supplied from the joint position information reception unit 22 in step S11 and multiple joints configuring a skeleton structure of a 3D character model stored in the 3D character model storage unit 21 with each other. Then, in a case where any joint is missing in the joint position information, the initial position deciding unit 23 estimates a position of the joint of the 3D character model missing in the joint position information. Consequently, the initial position deciding unit 23 decides the initial positions of all joints configuring the skeleton structure of the 3D character model and supplies a list of the joint IDs and the position data (x, y, z) of the joints to the rotation estimation unit 24 and the posture estimation updating unit 25.

In step S13, the rotation estimation unit 24 estimates rotation of end joint nodes (for example, the head, both hands, both feet, and so forth) in reference to the joint IDs and the position data (x, y, z) supplied from the initial position deciding unit 23 in step S12. Consequently, the rotation estimation unit 24 determines the roll rotation amount θ of the individual end joint nodes and supplies the joint ID and the roll rotation amount θ of the joint nodes to the posture estimation updating unit 25. It is to be noted that the roll rotation amount θ includes information indicative of a rotation direction (+direction or −direction).

In step S14, the rotation estimation unit 24 estimates rotation of the joint nodes of root nodes (for example, the waist, breast, and so forth) in reference to the joint IDs and the position data (x, y, z) supplied from the initial position deciding unit 23 in step S12. Consequently, the rotation estimation unit 24 determines the roll rotation amount θ of the individual joint nodes of the root nodes and supplies the joint IDs and the roll rotation amounts θ of the joint nodes of the root nodes to the posture estimation updating unit 25.

In step S15, the posture estimation updating unit 25 rotates all bones in the basic posture of the 3D character model in line with the roll rotation amount θ of the joint nodes of the root nodes supplied from the rotation estimation unit 24 in step S14, for example, of the joint node pelvis of the pelvis.

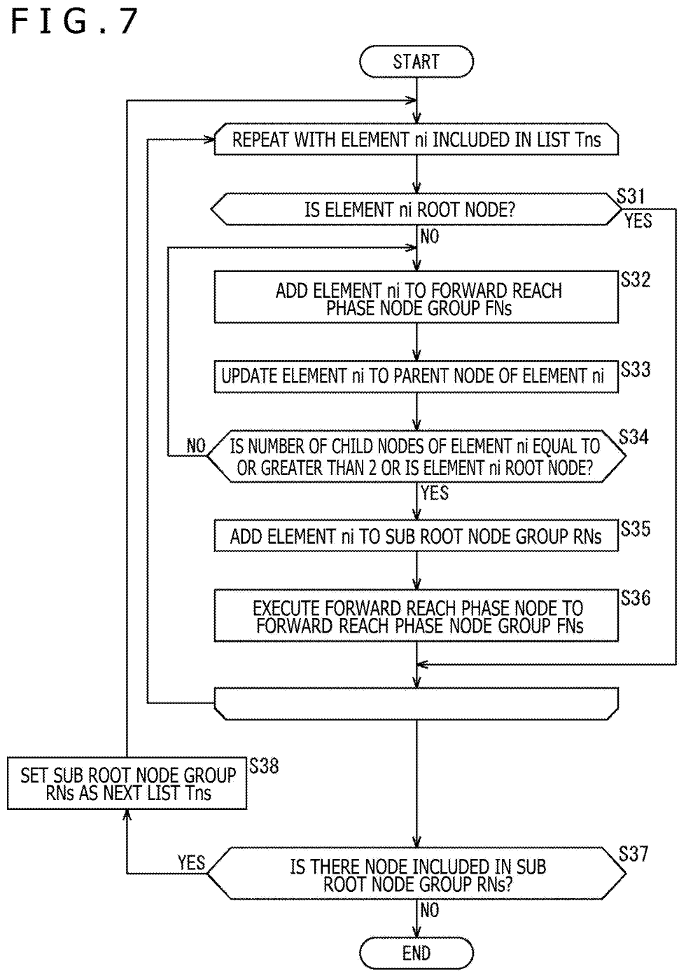

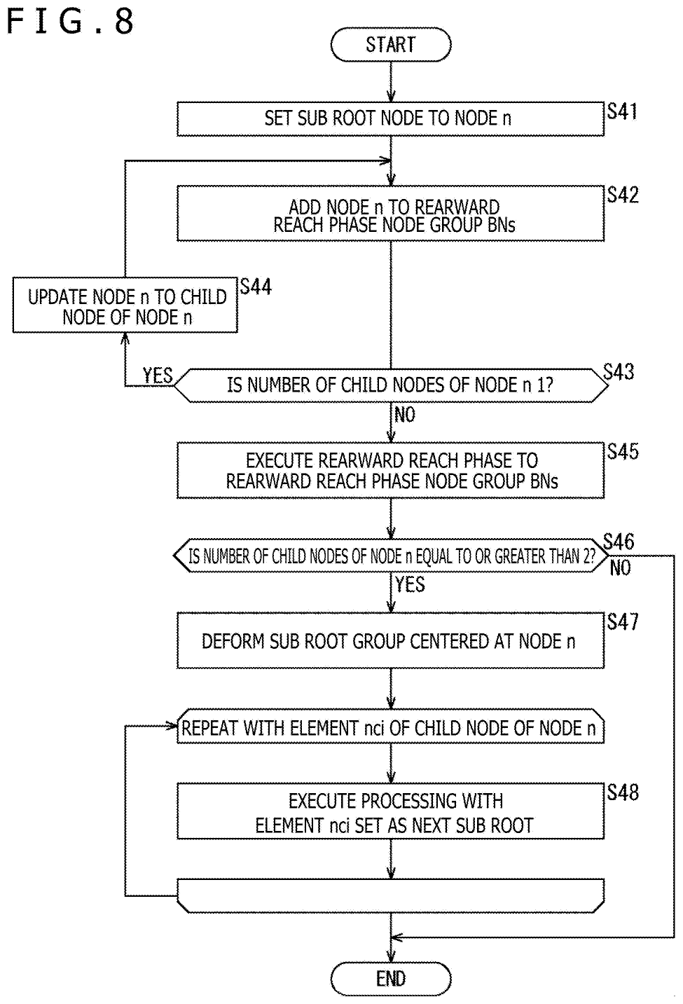

Thereafter, the posture estimation updating unit 25 executes a forward reach phase process (refer to FIG. 7 hereinafter described) in step S16, and executes a rearward reach phase and root group deformation process (refer to FIG. 8 hereinafter described) in step S17.

In step S18, the posture estimation updating unit 25 determines whether or not, as a result of repeated execution of steps S16 and S17, the difference between the pieces of position data (x, y, z) of the end joint nodes is equal to or smaller than a predetermined threshold value.

In a case where the posture estimation updating unit 25 determines in step S18 that the difference between the pieces of position data (x, y, z) of the end joint nodes is not equal to or smaller than the predetermined threshold value, the processing returns to step S16, and steps S16 and S17 are performed repeatedly.

On the other hand, in a case where the posture estimation updating unit 25 determines in step S18 that the difference between the pieces of position data (x, y, z) of the end joint nodes is equal to or lower than the predetermined threshold value, the processing advances to step S19. It is to be noted that, also in a case where the number of times by which steps S16 and S17 are repeated reaches a prescribed maximum number, that is, also in a case where steps S16 and S17 are performed repeatedly by the number of times equal to the maximum number of times, the processing can be advanced to step S19.

In step S19, the posture estimation updating unit 25 finally calculates the angle of all bones configuring the 3D character model in reference to the pieces of position data (x, y, z) of all joint nodes determined in step S17. Consequently, the posture estimation updating unit 25 estimates the posture of the whole body of the 3D character model and supplies, as the result of the calculation, the position data (x, y, z) and the rotation data (x, y, z, w) of all joint nodes to the rendering unit 26.

In step S20, the rendering unit 26 reflects the position data (x, y, z) and the rotation data (x, y, z, w) of all joint nodes supplied thereto from the posture estimation updating unit 25 in step S19 on the 3D character model read out from the 3D character model storage unit 21. By the reflection, the rendering unit 26 duplicates the posture according to the joint position information distributed thereto from the bone distribution server 13 on the 3D character model and outputs an image of a sports competition obtained by rendering the 3D character model from a desired free viewpoint.

FIG. 7 is a flow chart illustrating the forward reach phase process executed in step S16 of FIG. 6.

Processes from step S31 to step S36 are performed repeatedly with an element ni included in a list Tns.

In step S31, the posture estimation updating unit 25 determines whether or not the element ni is a root node, and in a case where the element ni is a root node, processes in steps S32 to S36 are skipped.

In a case where the posture estimation updating unit 25 determines in step S31 that the element ni is not a root node, the processing advances to step S32, in which the posture estimation updating unit 25 adds the element ni to a forward reach phase node group FNs.

In step S33, the posture estimation updating unit 25 updates the element ni added in step S32, to the parent node of the element ni.

In step S34, the posture estimation updating unit 25 determines whether or not the number of child nodes of the element ni is equal to or greater than 2 or whether or not the element ni is a root node.

In a case where the posture estimation updating unit 25 determines in step S34 that the number of child nodes of the element ni is not equal to or greater than 2 and the element ni is not a root node, the processing returns to step S32, and thereafter, similar processes are performed repeatedly. On the other hand, in a case where the posture estimation updating unit 25 determines in step S34 that the number of child nodes of the element ni is equal to or greater than 2 or that the element ni is a root node, the processing advances to step S35.

In step S35, the posture estimation updating unit 25 adds the element ni to a sub root node group RNs.

In step S36, the posture estimation updating unit 25 executes the forward reach phase for the forward reach phase node group FNs.

After such processes from step S31 to step S36 as described above are performed repeatedly with the element ni included in the list Tns, the processing advances to step S37.

In step S37, the posture estimation updating unit 25 determines whether or not there is a node included in the sub root node group RNs, and in a case where the posture estimation updating unit 25 determines that there is a node included in the sub root node group RNs, the processing advances to step S38.

In step S38, the posture estimation updating unit 25 decides the sub root node group RNs to be the next list Tns. Thereafter, the processing returns to the start, and similar processes are performed repeatedly for the next list Tns.

On the other hand, in a case where the posture estimation updating unit 25 determines in step S37 that there is no node included in the sub root node group RNs, the forward reach phase process is ended.

FIG. 8 is a flow chart illustrating the rearward reach phase and root group deformation process executed in step S17 of FIG. 6.

In step S41, the posture estimation updating unit 25 sets the sub root node to a node n.

In step S42, the posture estimation updating unit 25 adds the node n to a rearward reach phase node group BNs.

In step S43, the posture estimation updating unit 25 determines whether or not the number of child nodes of the node n is 1, and in a case where the number of child nodes of the node n is 1, the processing advances to step S44.

In step S44, the posture estimation updating unit 25 updates the node n to a child node of the node n. Thereafter, the processing returns to step S42, and thereafter, similar processes are performed repeatedly.

On the other hand, in a case where the posture estimation updating unit 25 decides in step S43 that the number of child nodes of the node n is not 1, the processing advances to step S45.

In step S45, the posture estimation updating unit 25 executes the rearward reach phase for the rearward reach phase node group BNs.

In step S46, the posture estimation updating unit 25 determines whether or not the number of child nodes of the node n is equal to or greater than 2, and in a case where the posture estimation updating unit 25 determines that the number of child nodes of the node n is not equal to or greater than 2 (that is, that the number of child nodes of the node n is less than 2), the processing is ended. On the other hand, in a case where the posture estimation updating unit 25 determines in step S46 that the number of child nodes of the node n is equal to or greater than 2, the processing advances to step S47.

In step S47, the posture estimation updating unit 25 deforms the sub root group centered at the node n.

The process in step S48 is performed repeatedly with an element nci of a child node of the node n. In step S48, the posture estimation updating unit 25 executes, deciding the element nci as a next sub root, the processes in and after step S41 (that is, the rearward reach phase and root group deformation process targeting the element nci).

After such a process in step S48 as described above is performed repeatedly with the element nci of the child node of the node n, the rearward reach phase and root group deformation process is ended.

By such a real time free viewpoint reproduction process as described above, even if there is a missing joint in the joint position information distributed from the bone distribution server 13 in regard to content distributed by real time free viewpoint distribution by the distribution system 11, the client terminal 14 can favorably reproduce the 3D character mode with a natural posture.

It is to be noted that such processes as described above with reference to FIGS. 7 and 8 are disclosed in detail in PTL 1 mentioned hereinabove.

### Example of Configuration of Computer

The series of processes (image processing method) described above can be executed not only by hardware but also by software. In a case where the series of processes is executed by software, a program that constructs the software is installed into a personal computer for universal use or the like.

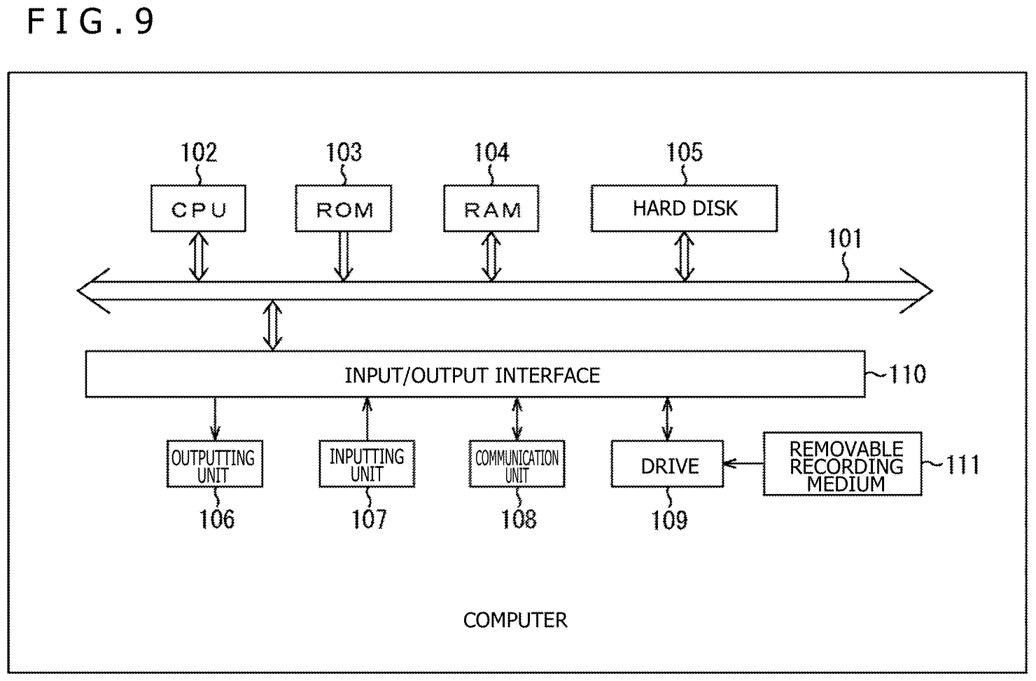

FIG. 9 is a block diagram depicting an example of a configuration of an embodiment of a computer into which a program for executing the series of processes described above is installed.

The program can be recorded in advance on a hard disk 105 or into a ROM 103 as a recording medium built in the computer.

Alternatively, the program can be stored (recorded) in advance into a removable recording medium 111 that is driven by a drive 109. Such a removable recording medium 111 as just described can be provided as what is generally called package software. Here, as the removable recording medium 111, for example, a flexible disk, a CD-ROM (Compact Disc Read Only Memory), an MO (Magneto Optical) disk, a DVD (Digital Versatile Disc), a magnetic disk, a semiconductor memory, and so forth are available.

It is to be noted that not only is it possible to install the program into a computer from such a removable recording medium 111 as described above but also it is possible to download the program into a computer through a communication network or a broadcasting network and install the program into the hard disk 105 built in the computer. In particular, it is possible to transfer the program by wireless transmission to the computer, for example, from a download site through an artificial satellite for digital satellite broadcasting or to transfer the program by wired transmission to the computer through such a network as the Internet.

The computer has a CPU (Central Processing Unit) 102 built therein, and an input/output interface 110 is connected to the CPU 102 through a bus 101.

If an inputting unit 107 is operated, for example, by the user to input a command to the CPU 102 via the input/output interface 110, then the CPU 102 executes the program stored in the ROM (Read Only Memory) 103 in accordance with the command. Alternatively, the CPU 102 loads the program stored in the hard disk 105 into a RAM (Random Access Memory) 104 and executes the program.

Consequently, the CPU 102 performs a process following the flow charts described hereinabove or a process to be performed by the configuration of the block diagram described hereinabove. Further, the CPU 102 causes a result of the process, for example, to be outputted from an outputting unit 106, transmitted from a communication unit 108, recorded on the hard disk 105, and so forth, for example, through the input/output interface 110 as occasion demands.

It is to be noted that the inputting unit 107 includes a keyboard, a mouse, a microphone and so forth. Meanwhile, the outputting unit 106 includes an LCD (Liquid Crystal Display), a speaker, and so forth.

Here, in the present specification, the processes performed in accordance with the program by the computer need not necessarily be performed in a time series in line with the order described as the flow charts. In particular, the processes performed in accordance with the program by the computer include processes that are executed in parallel or individually (for example, parallel processes or processes by an object).

Further, the program may be processed by a single computer (processor) or may be processed in a distributed manner by multiple computers. Moreover, the program may be transferred to and executed by a remote computer.

Further, in the present specification, the term system is used to signify an aggregation of multiple components (devices, modules (parts) and so forth), and it does not matter whether or not all components are accommodated in the same housing. Accordingly, multiple apparatuses accommodated in separate housings and connected to each other through a network are a system, and also one apparatus in which multiple modules are accommodated in a single housing is a system.

Further, for example, the configuration described as one apparatus (or one processing unit) may be divided to configure multiple apparatuses (or processing units). Conversely, the configurations described as multiple apparatuses (or processing units) in the foregoing description may be put together to configure a single apparatus (or processing unit). Further, a configuration not described hereinabove may naturally be added to the configuration of the individual apparatus (or processing units). Further, if a configuration or action of an entire system is substantially the same, then some of the configurations of a certain apparatus (or processing unit) may be included in the configuration of any other apparatus (or any other processing unit).

Further, for example, the present technology can assume a configuration of cloud computing by which one function is shared and cooperatively processed by multiple apparatuses through a network.

Further, for example, the program described above can be executed by any apparatus. In this case, it is sufficient if the apparatus has necessary functions (functioning blocks and so forth) and can acquire necessary information.

Further, for example, each of the steps described in the description of the flow charts can not only be executed by a single apparatus but also be shared and executed by multiple apparatuses. Moreover, in a case where multiple processes are included in one step, the multiple processes included in the one step can not only be executed by one apparatus but also be shared and executed by multiple apparatuses. In other words, it is also possible to execute multiple processes included in one step as a process of multiple steps. Conversely, it is also possible to execute a process described as multiple steps collectively as one step.

It is to be noted that, in a program to be executed by a computer, processes of steps that describe the program may be carried out in a time series in the order as described in the present specification or may be executed in parallel or executed individually at a necessary timing such as when the process is called. In short, the processes in the steps may be executed in an order different from the order described hereinabove if a contradiction does not occur. Further, the processes in the steps that describe this program may be executed in parallel with processes of any other program or may be executed in combination with processes of any other apparatus.

It is to be noted that the multiple present technologies in the present specification can be carried out alone and independently of each other if a contradiction does not occur. Naturally, desired ones of multiple present technologies can be carried out together. For example, it is also possible to carry out part or the entirety of the present technology described in connection with any of the embodiments in combination with part or the entirety of the present technology described in connection with a different one or ones of the embodiments. It is also possible to carry out any desired part or the entirety of the present technology described hereinabove together with some other technology that is not described hereinabove.

### Example of Combination of Configuration

It is to be noted that the present technology can also take the following configurations.
(1) An image processing apparatus including:

a deciding unit that estimates, in a case where, according to a result of comparison in corresponding relation between multiple pieces of joint information of an imaging target estimated from a captured image and multiple pieces of character model joint information of a desired character model, the multiple pieces of joint information and the multiple pieces of character model joint information are incoincident at least in part thereof, any one or more of the multiple pieces of character model joint information having no corresponding relation with the multiple pieces of joint information and decides all of the relevant pieces of character model joint information; and

a posture estimation unit that estimates a posture of a whole body of the character model, by using as initial positions, the decided relevant pieces of character model joint information.

(2) The image processing apparatus according to (1) above, in which,

in a case where the multiple pieces of joint information are less than the multiple pieces of character model joint information, the deciding unit estimates the character model joint information that is missing.
(3) The image processing apparatus according to (1) or (2) above, in which,

in a case where the multiple pieces of joint information are more than the multiple pieces of the character mode joint information, the deciding unit decides, as all of the relevant pieces of character model joint information, any one or more of the multiple pieces of joint information having a corresponding relation with the multiple pieces of character model joint information.
(4) The image processing apparatus according to any one of (1) through (3) above, in which

each of the multiple pieces of joint information indicates the number of joints and positions of the joints.
(5) The image processing apparatus according to any one of (1) through (4) above, in which

in a case where a length between joints according to the multiple pieces of joint information and a length between joints according to the multiple pieces of the character model joint information are different from each other, the deciding unit adjusts the length between the joints in the multiple pieces of joint information to adjust the length to the length between the joints in the multiple pieces of character model joint information and then decides the character model joint information.
(6) The image processing apparatus according to (5) above, in which

the character model and the imaging target are different in body proportion.
(7) The image processing apparatus according to any one of (1) through (6) above, further including:

a rotation estimation unit that estimates rotation information of an end joint node configuring a skeleton structure of the character model and a root node of a source of branching of the end joint node from a positional relation of other joint nodes and inputs the rotation information of the individual joint nodes to the posture estimation unit.
(8) The image processing apparatus according to (7) above, in which

the rotation information indicates a rotation direction and a rotation amount.
(9) The image processing apparatus according to (7) above, in which

the rotation estimation unit estimates postures of both hands of the imaging target in reference to the rotation information of fingers of both hands of the imaging target.
(10) The image processing apparatus according to (7) above, in which,

in a case where the rotation estimation unit is capable of acquiring information concerning a ball from data for sports, the rotation estimation unit estimates postures of both hands of the imaging object according to a position of the ball.
(11) The image processing apparatus according to any one of (1) through (10) above, in which

the character model includes a 3D character model including a three-dimensional skeleton structure of a character.
(12) The image processing apparatus according to (11) above, further including:

a rendering unit that applies the posture estimated by the posture estimation unit to a skeleton structure of the 3D character model to render the 3D character model from a desired free viewpoint and outputs the rendered 3D character model.
(13) An image processing method performed by an image processing apparatus, including:

estimating, in a case where, according to a result of comparison in corresponding relation between multiple pieces of joint information of an imaging target estimated from a captured image and multiple pieces of character model joint information of a desired character model, the multiple pieces of joint information and the multiple pieces of character model joint information are incoincident at least in part thereof, a corresponding one of the multiple pieces of character model joint information having no corresponding relation with the multiple pieces of joint information and deciding all of the pieces of character model joint information; and

estimating a posture of a whole body of the character model, by using, as an initial position, the decided character model joint information.
(14) A recording medium in which a program for causing a computer of an image processing apparatus to execute an image process is recorded, the image process including:

estimating, in a case where, according to a result of comparison in corresponding relation between multiple pieces of joint information of an imaging target estimated from a captured image and multiple pieces of character model joint information of a desired character model, the multiple pieces of joint information and the multiple pieces of character model joint information are incoincident at least in part thereof, a corresponding one of the multiple pieces of character model joint information having no corresponding relation with the multiple pieces of joint information and deciding all of the pieces of character model joint information; and

estimating a posture of a whole body of the character model, by using as an initial position, the decided character model joint information.

It is to be noted that the present embodiment is not limited to the embodiments described hereinabove and allows various alterations without departing from the subject matter of the present technology. Further, the advantageous effects described in the present specification are exemplary to the last and are not restrictive, and other advantageous effects may be available.

### REFERENCE SIGNS LIST

11: Distribution system

12: Capture system

13: Bone distribution server

14: Client terminal

21: 3D character model storage unit

22: Joint position information reception unit

23: Initial position deciding unit

24: Rotation estimation unit

25: Posture estimation updating unit

26: Rendering unit

1. An image processing apparatus comprising:
a deciding unit that estimates, in a case where, according to a result of comparison in corresponding relation between multiple pieces of joint information of an imaging target estimated from a captured image and multiple pieces of character model joint information of a desired character model, the multiple pieces of joint information and the multiple pieces of character model joint information are incoincident at least in part thereof, any one or more of the multiple pieces of character model joint information having no corresponding relation with the multiple pieces of joint information and decides all of the relevant pieces of character model joint information; and
a posture estimation unit that estimates a posture of a whole body of the character model, by using as initial positions, the decided relevant pieces of character model joint information.

2. The image processing apparatus according to claim 1, wherein,
in a case where the multiple pieces of joint information are less than the multiple pieces of character model joint information, the deciding unit estimates the character model joint information that is missing.

3. The image processing apparatus according to claim 1, wherein,
in a case where the multiple pieces of joint information are more than the multiple pieces of the character mode joint information, the deciding unit decides, as all of the relevant pieces of character model joint information, any one or more of the multiple pieces of joint information having a corresponding relation with the multiple pieces of character model joint information.

4. The image processing apparatus according to claim 1, wherein
each of the multiple pieces of joint information indicates the number of joints and positions of the joints.

5. The image processing apparatus according to claim 1, wherein,
in a case where a length between joints according to the multiple pieces of joint information and a length between joints according to the multiple pieces of the character model joint information are different from each other, the deciding unit adjusts the length between the joints in the multiple pieces of joint information to adjust the length to the length between the joints in the multiple pieces of character model joint information and then decides the character model joint information.

6. The image processing apparatus according to claim 5, wherein
the character model and the imaging target are different in body proportion.

7. The image processing apparatus according to claim 1, further comprising:
a rotation estimation unit that estimates rotation information of an end joint node configuring a skeleton structure of the character model and a root node of a source of branching of the end joint node from a positional relation of other joint nodes and inputs the rotation information of the individual joint nodes to the posture estimation unit.

8. The image processing apparatus according to claim 7, wherein
the rotation information indicates a rotation direction and a rotation amount.

9. The image processing apparatus according to claim 7, wherein
the rotation estimation unit estimates postures of both hands of the imaging target in reference to the rotation information of fingers of both hands of the imaging target.

10. The image processing apparatus according to claim 7, wherein,
in a case where the rotation estimation unit is capable of acquiring information concerning a ball from data for sports, the rotation estimation unit estimates postures of both hands of the imaging object according to a position of the ball.

11. The image processing apparatus according to claim 1, wherein
the character model includes a 3D character model including a three-dimensional skeleton structure of a character.

12. The image processing apparatus according to claim 11, further comprising:
a rendering unit that applies the posture estimated by the posture estimation unit to a skeleton structure of the 3D character model to render the 3D character model from a desired free viewpoint and outputs the rendered 3D character model.

13. An image processing method performed by an image processing apparatus, comprising:
estimating, in a case where, according to a result of comparison in corresponding relation between multiple pieces of joint information of an imaging target estimated from a captured image and multiple pieces of character model joint information of a desired character model, the multiple pieces of joint information and the multiple pieces of character model joint information are incoincident at least in part thereof, any one or more of the multiple pieces of character model joint information having no corresponding relation with the multiple pieces of joint information and deciding all of the relevant pieces of character model joint information; and
estimating a posture of a whole body of the character model, by using as initial positions, the decided relevant pieces of character model joint information.

14. A recording medium in which a program for causing a computer of an image processing apparatus to execute an image process is recorded, the image process comprising:
estimating, in a case where, according to a result of comparison in corresponding relation between multiple pieces of joint information of an imaging target estimated from a captured image and multiple pieces of character model joint information of a desired character model, the multiple pieces of joint information and the multiple pieces of character model joint information are incoincident at least in part thereof, any one or more of the multiple pieces of character model joint information having no corresponding relation with the multiple pieces of joint information and deciding all of the relevant pieces of character model joint information; and
estimating a posture of a whole body of the character model, by using as initial positions, the decided relevant pieces of character model joint information.
